(19) United States
(12) Patent Application Publication
(10) Pub. No.: US 2008/0086159 A1
(43) Pub. Date: Apr. 10, 2008

(54) Apparatus and method for reducing or eliminating the pain associated with an injection

(76) Inventor: Michael D. Zweifler, Maumelle, AR (US)

Correspondence Address: J. CHARLES DOUGHERTY, 200 WEST CAPITOL AVE, SUITE 2300, LITTLE ROCK, AR, 72201, US

(21) Appl. No.: 11/472,146

(22) Filed: Jun. 21, 2006

(51) Int. Cl. A61B 17/34

(52) U.S. Cl. 606/185

(57) ABSTRACT

An apparatus and method for facilitating painless or near-painless injections is described. The apparatus includes a guide that provides vibration around the site of the injection, thereby reducing the sensation of pain according to the pain gate theory. The guide also provides a reservoir to hold a bolus of anesthesia gel through the injection needle passes. The vibrational energy of the guide is also transferred to the needle itself, further reducing any pain associated with the injection. The guide may be sized to receive a needle recapping sheath, adding a safety enhancement feature to the device.

### BACKGROUND OF THE INVENTION

The present invention relates to a method and apparatus for performing medical and dental injections, and in particular to such a method and apparatus for performing such injections while reducing the pain associated with the injection.

Many people have a strong aversion to medical or dental injections performed by means of a needle due to the pain associated with the injection. This is a particularly pronounced problem with respect to injections made in the palate, due to the sensitivity of the palate and the common necessity of injecting anesthetics through the palate for various types of dental procedures. This aversion to receiving injections is known to lead many dental patients to either delay treatment, limit dental care to crisis or emergency situations, or avoid necessary dental treatment altogether. The result of such actions may be extensive tooth decay, tooth and bone loss, septic abscess, and in some extreme cases even death from systemic bacterial invasion of the patient's body.

Only two alternatives are available for dental patients that wish to avoid palate injections for these types of procedures. The first alternative is to forego anesthesia altogether, which is not likely to appeal to those patients who react adversely to the pain of the palatal injection. The other alternative, full or partial sedation dentistry, is very expensive, very time consuming, and entails all of the increased patient risks associated with general anesthesia. In addition, sedation dentistry requires that the patient bring another person to the dental visit who will be responsible for the patient's safe trip home. Neither of these alternatives are viable for most patients, and thus palatal injections remain the most common means of providing anesthesia for certain types of dental procedures, including nearly all upper dental arch procedures.

The current standard procedure for performing dental injections in the palate is to apply a topical anesthesia before the injection is performed. While this partially reduces the sensation of pain to the patient, the needle must travel far deeper into the patient's tissue than is reached by the topical anesthetic. Some dentists may also stimulate the patient's cheek or jaw in an effort to distract the patient from focusing his or her attention on the painful sensation of the needle penetrating the palate. Another approach used is the application of a cryogenic spray, which attempts to numb the area by rapidly cooling the skin surface.

The “pain gate” theory is a well known approach to understanding how the human body and brain process information about pain. The theory is based on the belief that small-diameter nerve fibers carry pain stimuli through a gate mechanism, while larger diameter nerve fibers going through the same gate can inhibit the transmission of the smaller nerves carrying the pain signal. Chemicals released as a response to the pain stimuli also influence whether the gate is open or closed for the brain to receive the pain signal. These observations led to the idea that the pain signals traveling to the brain can be interfered with by stimulating the periphery of the pain site, the appropriate signal-carrying nerves at the spinal cord, or particular corresponding areas in the brain stem or cerebral cortex.

It is generally recognised that the pain gate can be shut by stimulating those nerves responsible for carrying the touch signal. In particular, it has been recognized that those nerves in the area of the nerve that would otherwise send the pain signal may be stimulated through rubbing or the application of vibration in the area where pain would otherwise be sensed. In effect, the application of the additional stimuli at or near the localized area where pain would otherwise be sensed serves to “confuse” the nerve ending that would transmit the pain information. The pain information thus is not processed as such at the brain, and the patient does not sense the pain that would otherwise be associated with a needle injection or other painful stimuli.

The art includes a number of devices that are intended to reduce a patient's perceived pain resulting from an injection or other medical procedure. For example, U.S. Pat. No. 6,231,531 to Lum et al. teaches a blood sample device that surrounds the skin-piercing point with a vibrating surface that is held in contact with the patient's skin. Since the device is intended for drawing blood rather than injecting anesthesia or other medication, it does not include any means for delivering medication into the patient's tissue. It also does not include any means for applying a topical anesthetic to the skin where the puncture will occur, or any other pain-reduction mechanism in combination with the vibrating surface. The highly specialized and complex nature of the device would make it quite costly, and thus impossible to treat as disposable. Due to the risk of infection, any non-disposable device for performing injections is not likely to receive acceptance in the medical and dental communities.

U.S. Pat. No. 3,620,209 to Kravitz teaches a device to be used in conjunction with a traditional syringe injection for reducing the sensation of pain felt by the patient. The device comprises a horseshoe-shaped vibrational element that is strapped to the patient's body, roughly circumscribing the area where the injection will be made. As with the device taught by Lum et al, this device does not include any means for applying a topical anesthetic to the skin in the region where the injection will take place, or other means of pain reduction in combination with the vibrational element. In addition, this device would be unusable in areas such as the mouth where there is no means by which to strap the device at the area where the injection is to be performed.

The art also includes dental injection devices such as the “Vibraject” device, marketed by ITL Dental of Irvine, Calif. This device provides a vibration source that delivers vibrational energy to the needle used for the anesthetic injection. The vibrational energy source attaches directly to the syringe, thus transmitting vibration to the needle due to its direct mechanical attachment to the syringe. Such devices provide some stimulation of the area surrounding the point of injection, but the only source of vibration is the needle point itself. Thus while vibration of the needle may provide some desensitization, this is a limited effect and does not deliver pain-free or near pain-free injections in sensitive tissue areas such as the palate. In addition, the Vibraject device is bulky and cumbersome to use, which discourages dentists from using the device and may encourage patient fear simply due to the size of the apparatus.

None of the devices or methods described above are capable of effectively employing a combination of both topical anesthesia and nerve ending desensitization to maximize the reduction in pain sensation to the patient when an injection is performed. It is thus desirable to develop a method and apparatus for performing relatively pain-free injections using such a combination to maximum effect. Such method and apparatus should also be safe and economical to allow for widespread use by a variety of practitioners. In particular, any parts that contact the patient must be disposable in order to reduce the risk of cross-infection.

### SUMMARY OF THE INVENTION

The present invention is directed to a device that allows for pain-free or near pain-free injections, even in very sensitive tissues such as the palate tissue that must often be the site of anesthetic injections during dental procedures. The device comprises a guide in communication with a vibrational energy source. The needle of an ordinary disposable syringe may be inserted by the doctor or other practitioner through the guide to the site of the injection. The guide also comprises means for delivering topical anesthesia at the site of the injection. Preferably, the topical anesthesia used is in the form of a gel, which may form a bolus in a region of the guide designed to receive the gel. The needle passing through the guide passes through this bolus of topical anesthesia gel before reaching the tissue into which the injection is to be made. Since the needle is in contact with the guide during insertion, the needle also receives vibrational energy from the same source that provides this energy to the guide.

It may be seen that the invention provides pain reduction through three distinct mechanisms: needle vibration, topical anesthesia, and local tissue stimulation. The result is a pain-free or near pain-free injection, even in sensitive tissues. The guide may preferably be constructed of low-cost materials and may be considered disposable, thereby both lowering the cost of the method and enhancing the safety of the method through the reduction in opportunities for infection.

The use of a guide in the invention offers additional advantages in addition to those related directly to pain reduction. The guide may be sized and shaped such that it helps the practitioner place the syringe at the correct location and orientation for the injection, and may also help the practitioner monitor the depth of the injection for proper placement of the anesthesia or other medication. Such a guide is of particular value to a dentist, since dental injections occur in the mouth where precise placement and orientation of the injection needle is critical but may sometimes be difficult due to the limited space and visibility afforded by the mouth. Such a guide will be seen to be of particular value in this regard to those practitioners less experienced in performing these injections. In addition, the guide may in certain embodiments be designed to receive or embody a needle recapping device, which functions to prevent accidental needle puncture for the operator and any assistants.

It is therefore an object of the present invention to provide for a pain-free or near pain-free injection even in sensitive tissues such as the palate.

It is a further object of the present invention to provide a method and apparatus for providing pain-free or near pain-free injections at a low cost.

It is also an object of the present invention to provide for a method and apparatus for providing pain-free or near pain-free injections in a manner that is safe for the patient.

It is also an object of the present invention to provide for a method and apparatus for providing pain-free or near pain-free injections in a manner that is easy to learn and apply by a variety of doctors, dentists, and other practitioners.

It is also an object of the present invention to provide for a method and apparatus for providing pain-free or near pain-free injections whereby the injection may be made at most any location, without restriction due to the size and shape of the injection apparatus.

It is also an object of the present invention to provide for a method and apparatus for providing pain-free or near pain-free injections that does not require any additional specialized or expensive equipment for its use.

It is also an object of the present invention to provide for a method and apparatus for providing pain-free or near pain-free injections that may be employed by doctors, dentists, and other professionals accustomed to performing injections without specialized additional training.

It is also an object of the present invention to provide for a method and apparatus for providing pain-free or near pain-free injections that utilizes a standard disposable syringe and needle as commonly found in dental and medical practices today.

It is also an object of the present invention to provide a means for recapping a needle used for injections to prevent accidental needle puncture of the operator or any assistants.

These and other features, objects and advantages of the present invention will become better understood from a consideration of the following detailed description of the preferred embodiments and appended claims in conjunction with the drawings as described following:

### DESCRIPTION OF THE DRAWINGS

FIG. 1 is a partial cut-away view of a preferred embodiment of the present invention in conjunction with a detail of a patient's mouth where the invention may be employed.

FIG. 2A is a partial cut-away view of a first variation in the design of a needle guide in conjunction with a preferred embodiment of the present invention.

FIG. 2B is a partial cut-away view of a second variation in the design of a needle guide in conjunction with a preferred embodiment of the present invention.

FIG. 2C is a partial cut-away view of a third variation in the design of a needle guide in conjunction with a preferred embodiment of the present invention.

FIG. 2D is a partial cut-away view of a fourth variation in the design of a needle guide in conjunction with a preferred embodiment of the present invention.

FIG. 2E is a partial cut-away view of a fifth variation in the design of a needle guide in conjunction with a preferred embodiment of the present invention.

FIG. 2F is a partial cut-away view of a sixth variation in the design of a needle guide that may be held by the patient, in conjunction with a preferred embodiment of the present invention.

FIG. 3 is a cut-away view of a universal adapter for an extra-oral vibrating device (EVD) according to a preferred embodiment of the present invention.

FIG. 4A is a partial cut-away view of a universal adapter in conjunction with a first EVD according to a preferred embodiment of the present invention.

FIG. 4B is a partial cut-away view of a universal adapter in conjunction with a second EVD according to a preferred embodiment of the present invention.

FIG. 4C is a partial cut-away view of a universal adapter in conjunction with a third EVD according to a preferred embodiment of the present invention.

FIG. 5 is a partial cut-away view of an alternative preferred embodiment of the present invention in conjunction with a detail of a patient's mouth where the guide opening is designed to receive a needle recapping sheath.

### DETAILED DESCRIPTION OF THE PREFERRED EMBODIMENTS

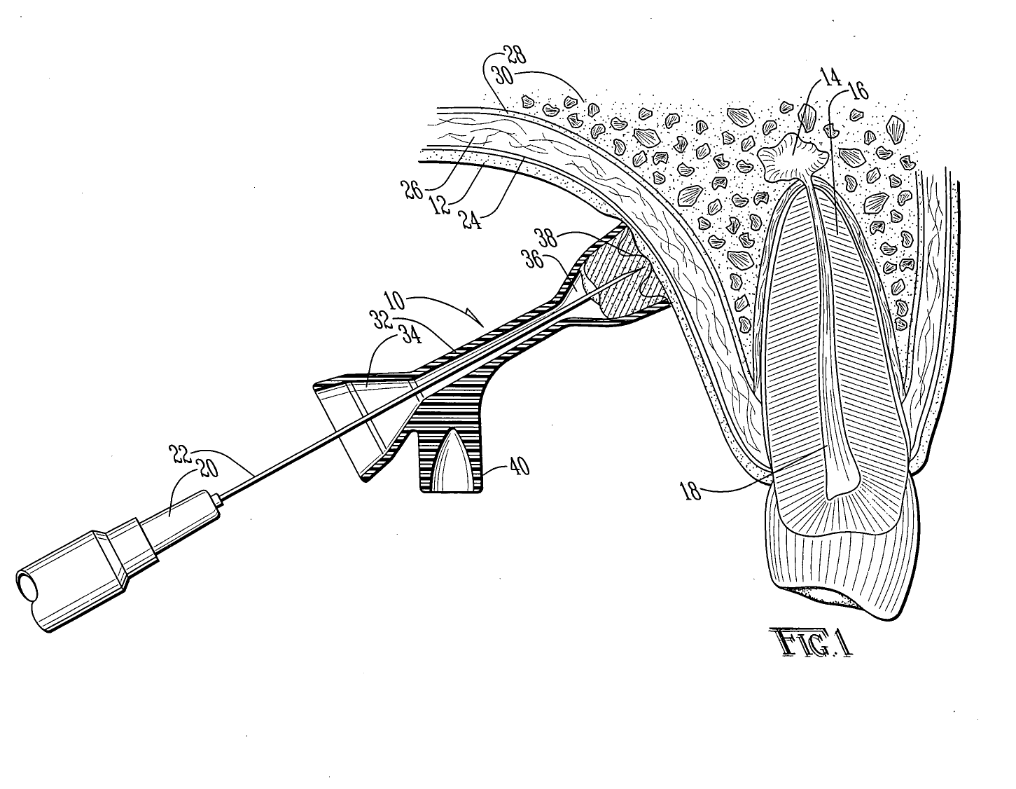

With reference to FIG. 1, a preferred embodiment of the present invention may be described. Guide 10 is shown in position at palatal tissue 12 of a patient's mouth. The purpose of the preferred embodiment is to enable a dentist to inject anesthesia through palatal tissue 12 in order to anesthetize target area 14 at the root of tooth 16 with nerve 18. Such anesthetization is necessary in order for the dentist to perform a dental procedure such as tooth reduction for crown preparation, or decay removal including profound anesthesia in the case of extraction or root canal (endodontic) procedures. Other potential uses could include injecting the gingiva only as in the case of periodontal (soft tissue) procedures, or actual block anesthesia into or around foramina containing a nerve bundle, i.e., the posterior palatal or mental nerve blocks. In the case of tooth reduction as illustrated in FIG. 1, guide 10 is used to lead needle 22 on syringe 20 through channel 32 to pierce palatal tissue 12, connective tissue 24, interstitial tissue 26, periosteum 28 (if necessary), and cancellous bone layer 30 to reach target area 14.

Guide 10 may be constructed of any sufficiently strong material, but is preferably formed of an acrylic plastic. The material chosen should ideally be suitable for inexpensive manufacture in order to be disposable. Alternatively, guide 10 may be constructed of a material that is designed to withstand autoclave sterilization, but this is less desirable since it is believed that a non-disposable product will not receive acceptance in the medical and dental community. It is preferred that guide 10 be constructed of a transparent or semi-transparent material such that the practitioner can see the progress of needle 22 through main channel 32 of guide 10 during the insertion procedure and during the injection.

Guide 10 preferably includes a flared syringe opening 34 that is wider than the main body of channel 32. The purpose for flared opening 34 is to more easily receive needle 22 and guide it into channel 32. Flared opening 34 allows less precision to be used when needle 22 is inserted, thereby increasing the ease and speed with which the preferred embodiment may be used during a dental procedure. Flared opening 34 also reduces the likelihood that an accidental needle prick may occur due to the operator accidentally missing guide 10 while attempting to insert needle 22.

In certain of the preferred embodiments, flared syringe opening 34 of guide 10 may be expanded and lengthened as illustrated in FIG. 5 to provide a needle recapping aid. In these embodiments, opening 34 is sized to receive needle recapping sheath 50, which may be of a standardize shape and size as are known in the art for disposing of used needles safely. Preferably, opening 34 in this embodiment includes grooves 52 to receive ridges 54 extending longitudinally down the length of sheath 50. Upon the completion of an injection, needle 22 may be removed from guide 10, sheath 50 may be inserted, and needle 22 may then be inserted into sheath 50 for recapping. By using guide 10 in this manner, the risk to the operator or an assistant who is attempting to recap needle 22 is greatly reduced, since guide 10 provides a hand grip that allows such person to keep his or her hand well away from the point of needle 22 during the recapping process. The lip of opening 34 also serves to trap the tip of needle 22 should such person miss the opening of sheath 50 during the recapping process. Preferably, guide 10, sheath 50, and needle 22 may be disposed of as a unit when recapping is complete.

Returning now to FIG. 1, guide 10 also preferably includes a bowl 36 at the end opposite flared opening 34 to receive topical anesthesia. This anesthesia is preferably in the form of a gel, which will form a bolus when placed within bowl 36. Needle 22 must pass through this bolus during insertion, and thus will carry topical anesthesia at its tip when inserted into palatal tissue 12. The presence of the bolus of anesthesia at bowl 36 also ensures that topical anesthesia is applied to the portion of palatal tissue 12 in the area immediately surrounding the location at which needle 22 will penetrate palatal tissue 12.

In the preferred embodiment, any common sort of syringe 20 and needle 22 may be employed with guide 10. Preferably, syringe 20 and needle 22 are of the disposable type as widely used in medical and dental practices today. These are available in numerous sizes for various tasks. The length of guide 10 may be matched to the length of needle 22 to ensure that needle 22 can pass far enough through guide 10 that needle 22 may penetrate tissue to the proper depth.

Connector 40 serves to provide a mechanical connection between guide 10 and an extra-oral vibrating device (EVD). This device may be any source of mechanical vibration that causes a like vibration at guide 10. The EVD preferably operates at ultrasonic frequencies. In one preferred embodiment, the EVD may be a handpiece such as the powered base unit from an Oral-B Hummingbird flossing unit made by the Gillette Company of Boston, Massachusetts. Many other vibrational sources could be used as EVDs in alternative embodiments.

The vibration imparted to guide 10 through connector 40 travels to nubs 38 at the outer edge of bowl 36. These nubs are held in contact with palatal tissue 12 when the preferred embodiment is in use, and thus the vibrational energy imparted to nubs 38 is directed into the tissue. In accordance with the pain gate theory, the massaging effect of nubs 38 at palatal tissue 12 serves to lessen or eliminate the sensation of pain when needle 22 pierces palatal tissue 12 within the area that is being stimulated by nubs 38. Alternatively, nubs 38 could be replaced by a smooth surface at the edge of bowl 36, or a smoothly serrated or grooved surface.

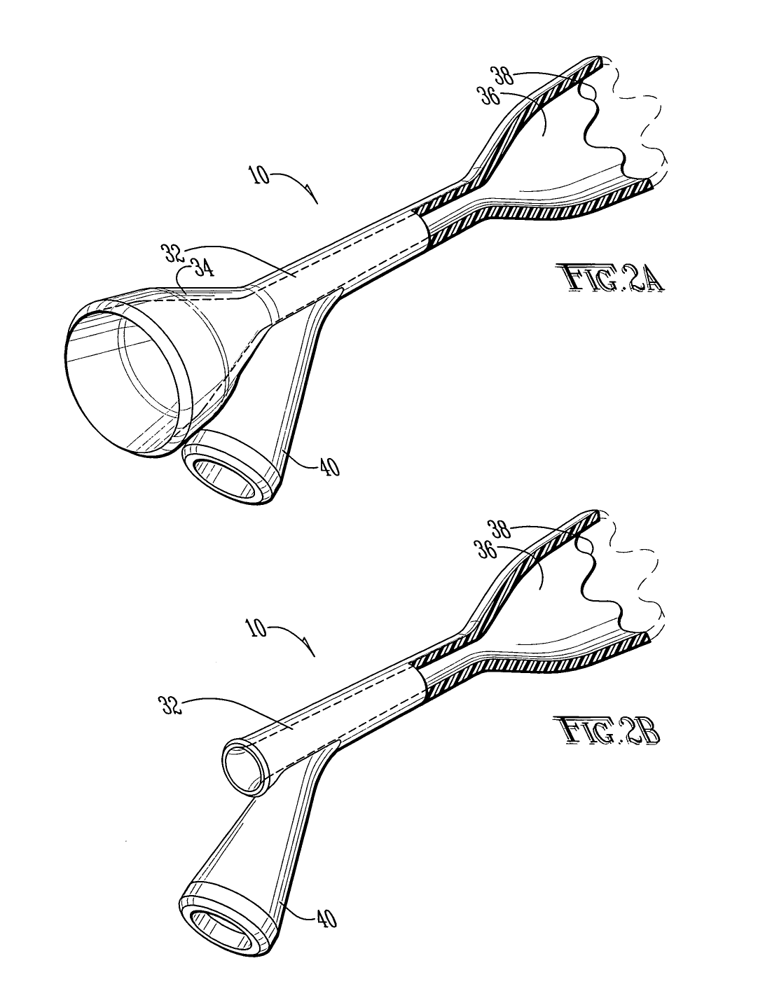

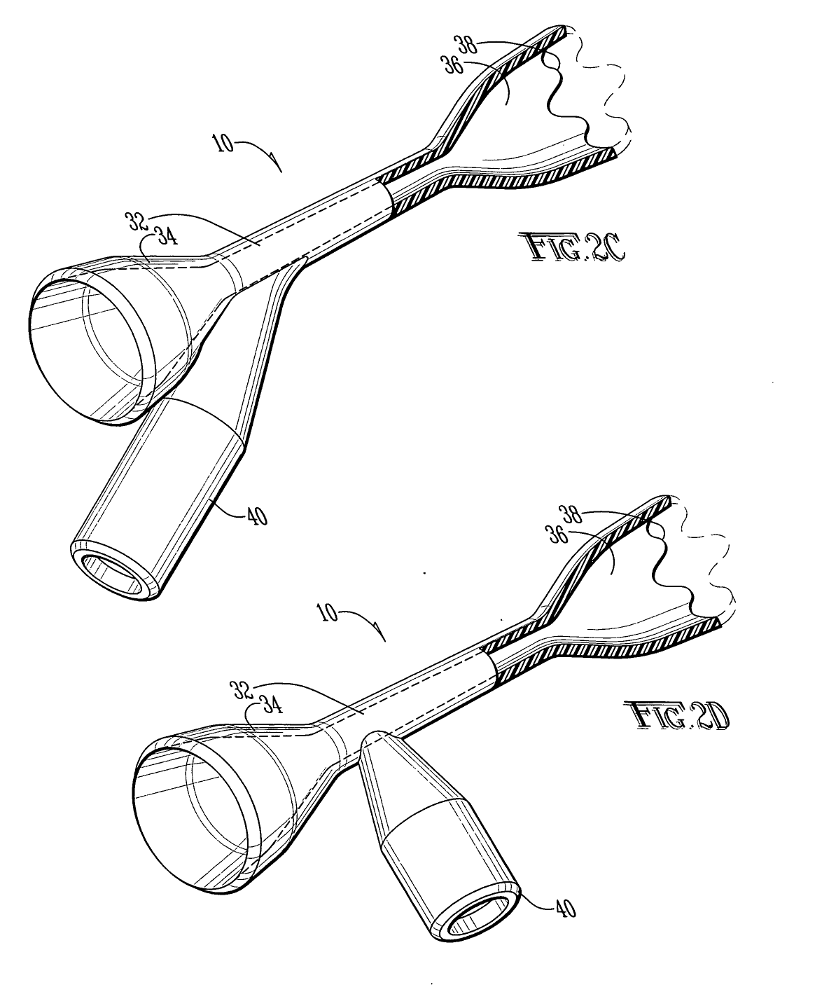

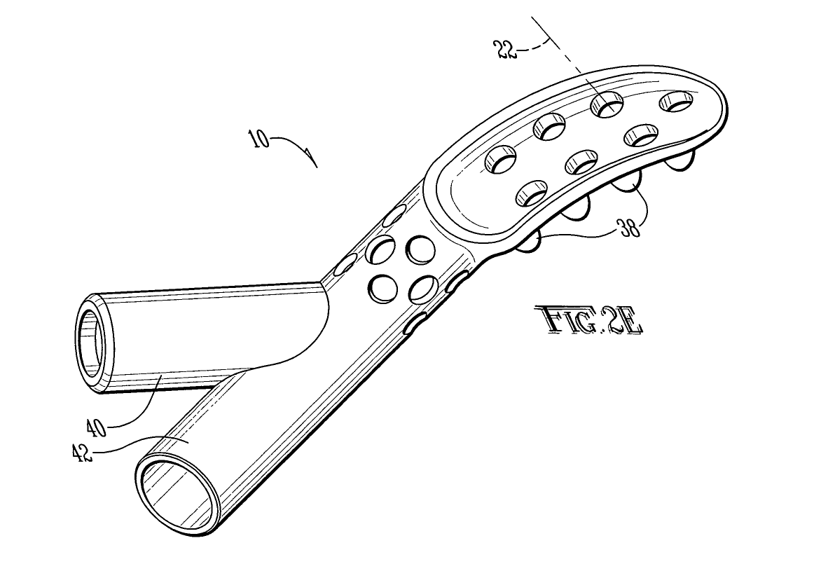

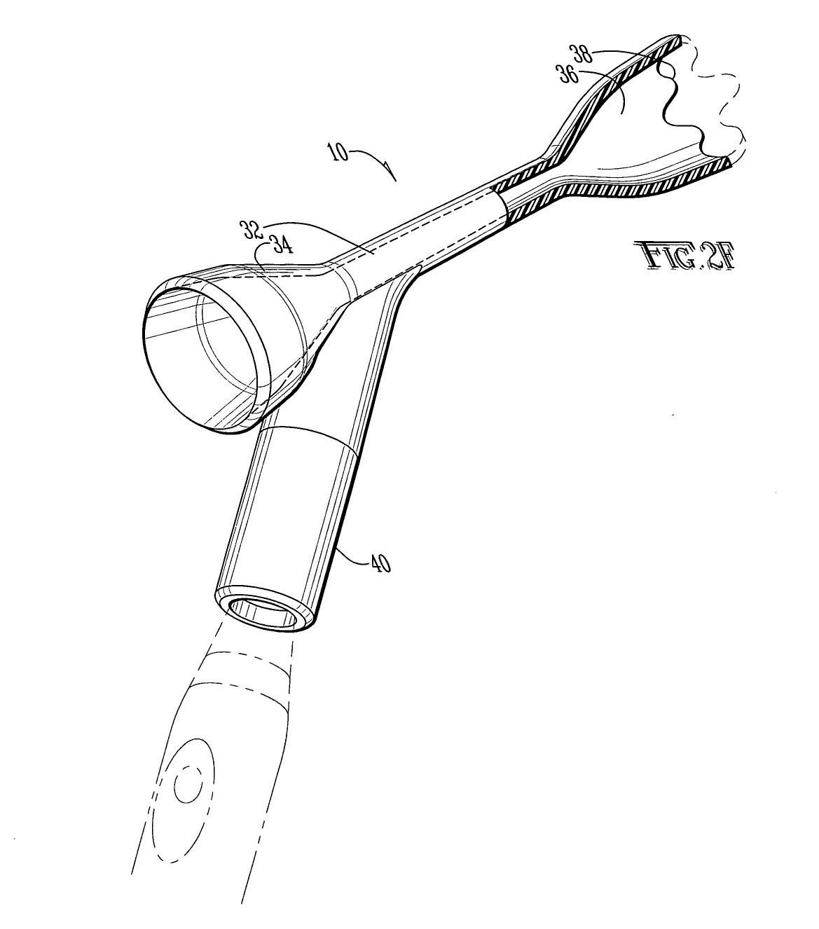

Referring now to FIGS. 2A through 2F, some alternative preferred embodiments of guide 10 are shown in various shapes. These different shapes may be employed for different types of injections, thereby aiding the delivery of anesthesia to the precise point in the mouth where the medication is required. The development of various shapes for guide 10 further allows for ease in orienting needle 22 to conform to different sites in the mouth, including but not limited to the hard palate, soft palate, inferior alveolar block anesthesia, and posterior, middle, and superior alveolar injection. The various shapes of guide 10 may also be used for intra- or extra-oral anesthesia as in the case of irritational fibroma removal at the vermillion border of the lip. These are examples for illustration only, and the invention may include any number of other specific shapes for various dental and medical injections.

In the alternative embodiment of FIG. 2E, an additional suction attachment 42 is provided through which a suction hose may be connected. The suction hose is preferably of a type as is commonly found in dental offices and may be attached for various procedures. Suction is used, for example, to remove rinsing fluid from the mouth during some dental procedures.

In FIG. 2F, a specialized EVD handle is shown. This type of EVD is of a form as commonly seen in the handle of powered toothbrushes. In this form, the device may be employed by a dentist that wishes to allow a patient to exercise a greater amount of control during the procedure. It is believed by the inventor that the anxiety level of some patients may be significantly reduced by involving the patient in his or her own dental procedure. By acquiring a sense of control over the injection process, the patient's perceived pain may also be reduced. Using the preferred embodiment of FIG. 2F, the patient may hold the apparatus at the handle while under the instruction and control of the dentist, thereby providing a sense of involvement in his or her own treatment. This involvement may also serve to further distract the patient from the actual injection of anesthesia by means of needle 22 since the patient is concentrating on following the dentist's instructions in properly holding guide 10 in place with the handle.

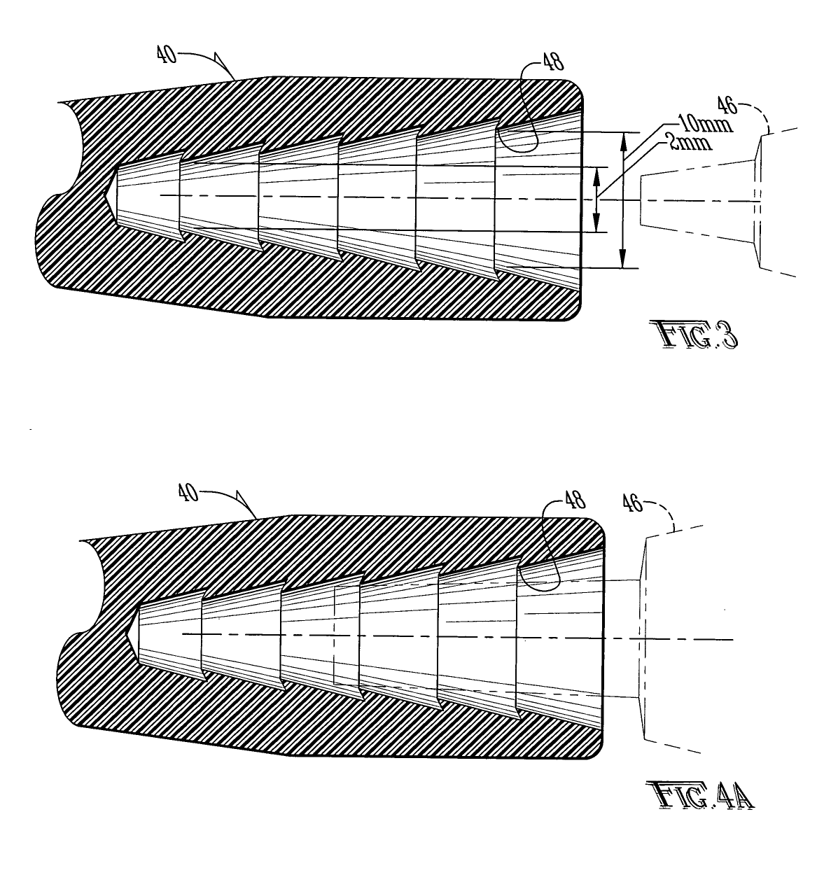

Referring now to FIG. 3, a particular embodiment of connector 40 is shown for a preferred embodiment of the present invention. This version of connector 40 is a “universal” connector designed to allow guide 10 to be employed easily and interchangeably with a variety of vibrational energy sources. Connector 40 includes a hollow, roughly cone-shaped portion that includes a series of barbed or “shark-tooth” projections 48. These projections 48 are pointed rearward toward guide 10, such that any device inserted into the hollow portion of connector 40 is held in place once inserted. Preferably, projections 48 are sufficiently rigid to hold an EVD device 46 in place with respect to guide 10, but sufficiently resilient such that a firm application of rearward force by the operator can displace EVD device 46 from connector 40. Many plastics would be appropriate as a material for the fabrication of connector 40 and projections 48.

In the preferred embodiment, projections 48 are positioned around the circumference of the hollow portion of connector 40 such that EVD devices 46 of various diameters may be engaged at connector 40. This is the reason that this embodiment of connector 40 is considered to be a “universal” connector. For example, a first row of projections 48 may provide an opening of 10 mm, a second row of projections 48 an opening of 8 mm, a third row of projections 48 an opening of 6 mm, a fourth row of projections 48 an opening of 4 mm, and a fifth row of projections 48 an opening of 2 mm. An EVD 46 with an engagement tip having a diameter of 4 mm, for example, will pass the first, second, and third rows without engagement, but will be held in place when it reaches the fourth row during insertion into connector 40. The device may be removed by pulling firmly in order to overcome projections 48. Another EVD device 46 may be inserted with an engagement tip having a diameter of 8 mm, which will engage at the second row of projections 48. In an alternative embodiment not shown, projections 48 may be replaced by a raised ridge or groove that spirals outwardly along the surface of the hollow portion of connector 40. It should be noted that non-universal means of engaging EVD devices with connector 40, such as threads and ball-lock arrangements as are known in the art, are also within the scope of the invention.

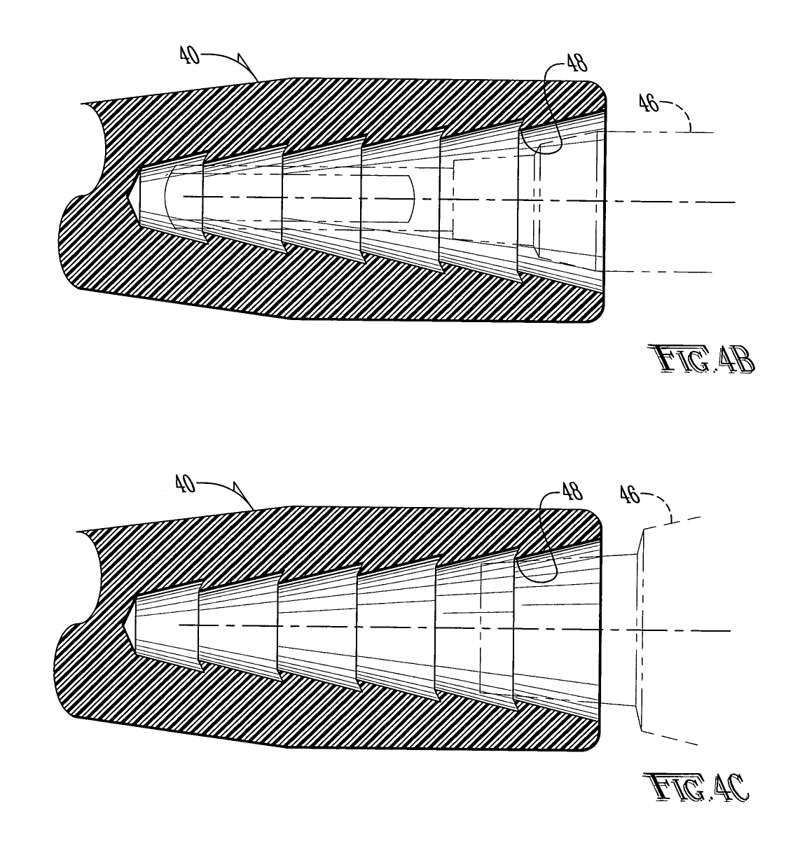

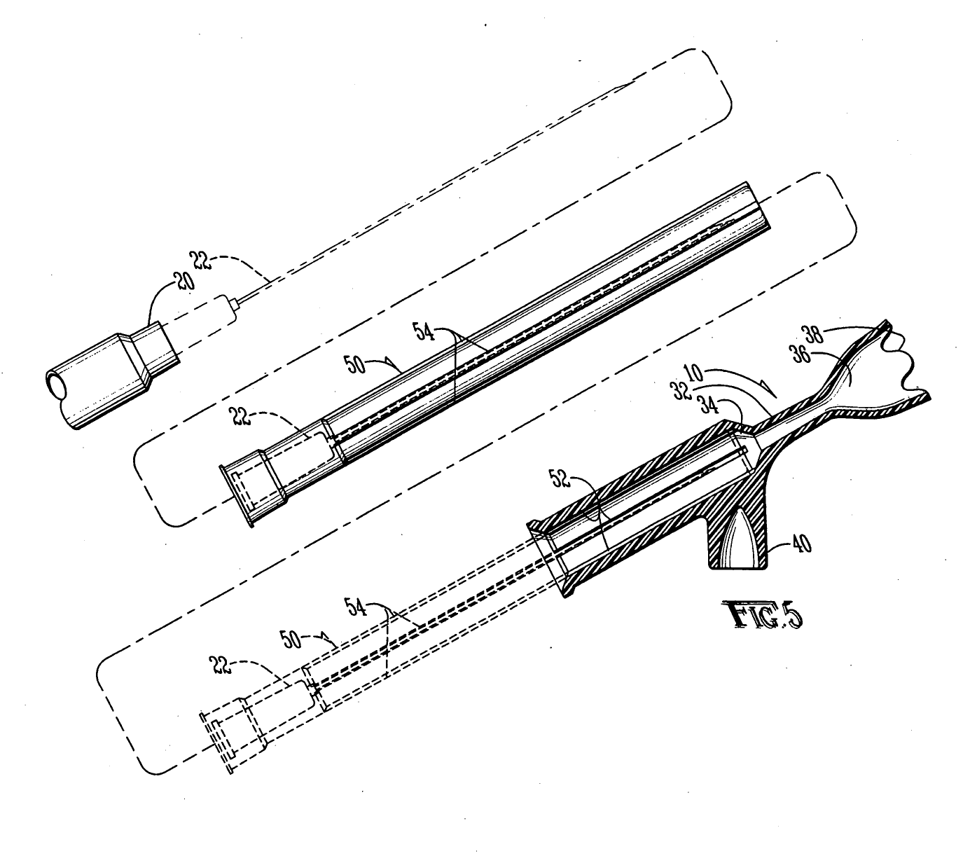

Referring now to FIGS. 4A through 4C, different specific EVD devices are depicted in use with universal connector 40 as shown in FIG. 3. In FIG. 4A, EVD device 46 is an Oral-B Hummingbird flosser hand unit. This device has a head circumference of roughly 6-9 mm. In FIG. 4B, EVD device 46 is any of the many types of electric toothbrushes available on the commercial market. The bristles of the toothbrush body have been removed for use with connector 40. The head of such toothbrushes typically is pointed, having a size in the range of 1-5 mm. FIG. 4C depicts EVD device 46 as the power unit from a RotoDent professional toothbrush, which is available from Prodentec of Batesville, Ark. The drive head of the power unit on this device is about 10 mm wide.

The operation of the preferred embodiment of the invention may now be described, again with reference to FIG. 1 in the context of a dental procedure. The dentist first identifies target area 14 to which anesthesia must be directed. If multiple shapes or sizes of guide 10 are available, as shown, for example, in FIGS. 2A through 2F, the dentist then selects the appropriate guide 10 from among those available for this particular procedure. EVD device 36 is attached, using whichever such device is desired by the dentist. Depending upon the type of connector 40 in use, EVD device 36 may be snapped, threaded, or simply pushed into place at connector 40.

Guide 10 is next loaded with topical gel anesthesia at bowl 36. This loading procedure is preferably performed by cotton swab or spatula. The anesthesia used may be of any type commonly employed for topical anesthesia in dental procedures, such as lidocaine, or a blend of various such compounds. In one preferred embodiment, the topical anesthetic gel comprises 20% by volume lidocaine, 20% by value prilocaine, and 8% by volume tetracaine. After loading, EVD device 36 is switched to the “on” position and guide 10 is placed in the patient's mouth such that nubs 38 are in contact with palatal tissue 12. Gentle pressure should be applied downwardly by the dentist such that nubs 38 make secure contact with palatal tissue 12. Guide 10 is then aligned such that channel 32 is directly in line with the point at which needle 22 is to pierce palatal tissue 12 for the injection. Syringe 20, which has prior to the procedure been loaded with the appropriate anesthesia in the usual manner, is then directed toward guide 10 such that needle 22 enters flared opening 34 and passes through channel 32. Upon entering bowl 36, the beveled tip of needle 22 will pick up anesthesia gel from the bolus of material present in bowl 36, and carry it forward as the tip of needle 22 contacts palatal tissue 12. Since guide 10 in the preferred embodiment is transparent or semi-transparent, the dentist may monitor the progress of needle 22 through guide 10 visually during this operation.

It may be seen that as the tip of needle 22 reaches palatal tissue 12, there are three separate mechanisms in play by which any perception of pain by the patient is being relieved or avoided. First, the ultrasonic vibration of nubs 38 at the surface of palatal tissue 12 provides a loss of pain sensation as contemplated by the pain gate theory. Second, needle 22, by virtue of its contact with guide 10, is also picking up vibrational energy from EVD 36, and the vibration of the point of needle 22 at the point where it pierces palatal tissue 12 also serves to lessen the sensation of pain due to needle 22. Finally, the design of guide 10 forces needle 22 to pick up topical anesthetic gel before and during the time that it pierces palatal tissue 12, such that anesthetic is always carried just at the tip of needle 22. As needle 22 passes through palatal tissue 12 and into the deeper tissues, the dentist may provide gentle pressure to release anesthetic from syringe 20 through needle 22 into the tissue. The tissue is thus being anesthetized just before the needle reaches the tissue that will be pierced.

It may be seen that the preferred embodiment is designed in such a manner that the three different pain-alleviating effects described above are all accomplished with one motion of the operator. This motion is in conformance with the standard procedures in use by dentists today, and therefore requires no specialized training in order to be successfully adopted into a dental practice.

The present invention has been described with reference to certain preferred and alternative embodiments that are intended to be exemplary only and not limiting to the full scope of the present invention as set forth in the appended claims. In particular, and for purpose of example only, the preferred embodiment has been described for use in connection with a palatal injection performed as part of a dental procedure. The invention is not so limited, and could, for example, be used for a variety of other dental and medical procedures that involve an injection using a needle.

I claim:

1. An injection guide, said guide comprising:
(a) a main channel sized to receive a needle;
(b) a reservoir in communication with said main channel and adapted to receive a topical anesthesia wherein a needle passing through said main channel may pass through said reservoir;
(c) a connector sized to receive a vibrational energy source; and
(d) a tissue contact surface adapted to contact tissue into which the injection is to be made.

2. The guide of claim 1, wherein said contact surface comprises an irregular surface.

3. The guide of claim 2, wherein said contact surface comprises a plurality of nubs.

4. The guide of claim 1, further comprising a flared opening in communication with said main channel, wherein at least a portion of said flared opening is of a larger circumference than said main channel.

5. The guide of claim 4, further comprising a needle sheath, and wherein said flared opening is sized to removably receive said needle sheath.

6. The guide of claim 1, wherein at least a portion of said reservoir is of a larger circumference than said main channel.

7. The guide of claim 6, wherein said reservoir is open at a distal end of the guide, and said contact surface comprises a rim at said distal end of said guide.

8. The guide of claim 1, wherein said connector comprises an opening sized to receive one of a plurality of vibrational energy sources of varying diameters.

9. The guide of claim 8, wherein said connector comprises an inner surface comprising a plurality of projections.

10. The guide of claim 9, wherein said connector comprises a distal end, and said plurality of projections comprises a plurality of opposing sets of projections, wherein the distance between opposing sets of projections is greater for those of said opposing sets of projections closer to said distal end of said connector.

11. The guide of claim 8, wherein said connector comprises an inner surface comprising one of a spiraling slot and a spiraling ridge.

12. The guide of claim 1, further comprising a vibrational energy source comprising an ergonomic handle.

13. A method of reducing pain associated with an injection into a tissue using a needle, comprising the method steps of:
(a) attaching a vibrational energy source to a needle guide, wherein the needle guide comprises a main channel, a reservoir in communication with the main channel, and a contact surface;
(b) applying a topical anesthetic within the reservoir of the needle guide;
(c) placing the needle guide contact surface against the tissue to impart vibration to the tissue;
(d) inserting the needle through the main channel and into the reservoir such that a tip of the needle receives topical anesthetic from within the reservoir; and
(e) inserting the needle into the tissue.

14. The method of claim 13, wherein the vibrational energy source provides vibrational energy in the ultrasonic frequency range.

15. The method of claim 14, wherein the needle contacts the guide such that vibrational energy is imparted to the needle from the guide.

16. The method of claim 13, further comprising the step of applying downward pressure to the guide wherein the contact surface makes firm contact with the tissue in order to impart vibrational energy to the tissue.

17. An injection kit, said kit comprising:
(a) a needle guide comprising a channel;
(b) a vibrational energy source connected to said guide;
(c) a syringe; and
(d) a needle attached to said syringe, wherein said needle is sized to fit within said channel of said guide.

18. The kit of claim 17, wherein said needle guide further comprises a reservoir in communication with said channel.

19. The kit of claim 18, wherein said guide further comprises a contact surface, wherein said needle may pass through said channel and said reservoir and extend through said contact surface.

20. The kit of claim 19, wherein said contact surface is irregular.

21. The kit of claim 20, wherein said contact surface comprises a plurality of nubs.

22. The kit of claim 17, further comprising a needle sheath, and wherein at least a portion of said channel of said needle guide is sized to receive said needle sheath.
